(19) United States
(12) Patent Application Publication
(10) Pub. No.: US 2024/0164672 A1
(43) Pub. Date: May 23, 2024

(54) STRESS DETECTION

(71) Applicant: Apple Inc., Cupertino, CA (US)

(72) Inventors: Brian Pasley, Berkeley, CA (US); Grant H. Mulliken, Los Gatos, CA (US)

(21) Appl. No.: 18/283,849

(22) PCT Filed: Mar. 16, 2022
(86) PCT No.: PCT/US2022/020494

§ 371 Date: Sep. 25, 2023

Related U.S. Application Data
(60) Provisional application No. 63/168,623, filed on Mar. 31, 2021.
(60) Provisional application No. 63/221,061, filed on Jul. 13, 2021.

(51) Int. Cl. A61B 5/16; A61B 5/00

(52) U.S. Cl. CPC A61B 5/165; A61B 5/163; A61B 5/486; A61B 5/6898; A61B 5/7267; A61B 2560/0242

(57) ABSTRACT

Various implementations disclosed herein include devices, systems, and methods that determine a stress level of a user during presentation of content. For example, an example process may include obtaining physiological data associated with a user during an experience in an environment, determining a context of the experience based on sensor data of the environment, determining a stress level of the user during a portion of the experience based on the obtained physiological data and the context of the experience, and providing a feedback mechanism during the experience based on the stress level.

### TECHNICAL FIELD

The present disclosure generally relates to presenting content via electronic devices, and in particular, to systems, methods, and devices that determine a user's stress level during and/or based on the presentation of electronic content and the environment of the user.

### BACKGROUND

A user's stress level while viewing and/or listening to content on an electronic device can have a significant effect on the user's experience. For example, stress awareness may help facilitate more meaningful experiences, such as watching educational or entertaining content, learning a new skill, reading a document, playing a video game, or social interactions. Improved techniques for assessing the stress levels of users viewing and interacting with content may enhance a user's enjoyment, comprehension, and learning of the content. Moreover, content may not be presented in a way that mitigates stress to a particular user. Content creators and systems may be able to provide better and more tailored user experiences that a user is more likely to enjoy, comprehend, and learn from based on stress level information.

### SUMMARY

Various implementations disclosed herein include devices, systems, and methods that assess physiological data of a user (e.g., gaze characteristic(s)) and the context of an experience of the user to predict a stress level (e.g., predicting when a user is experiencing stress), and provide a feedback mechanism based on the stress level of the user (e.g., a notification to the user). Different feedback mechanisms can be presented based on the user's stress levels and the type of stress. For example, a feedback mechanism may provide simple notifications, recommend meditation, or create a relaxing VR space to encourage a reduced stress level. Aggregated data may be used for stress training programs (e.g., if a user meditates/exercises every morning, the system may quantify how much doing so is helping with stress levels).

Physiological data may be used to determine stress level. For example, some implementations may identify that the user's eye characteristics (e.g., blink rate, stable gaze direction, saccade amplitude/velocity, and/or pupil radius), electrodermal activity, heart rate, and/or movement, correspond to a “calm” stress level rather than a “distressed” stress level.

Context may additionally be used to determine stress level. For example, a scene analysis of an experience can determine a scene understanding of the visual and/or auditory attributes associated with content being presented to the user (e.g., what is being presented in video content) and/or attributes associated with the environment of the user (e.g., where is the user, what is the user doing, what objects are nearby). These attributes of both the presented content and environment of the user can improve the determination of the user's stress level.

Some implementations improve stress level assessment accuracy, e.g., improving the assessment of a user's stress level. Some implementations improve a user experience by providing a notification based on an identified stress level (e.g., notifying the user they are exhibiting higher than normal stress levels during a stressful experience). Some implementations improve user experiences by providing stress level assessments that minimize or avoid interrupting or disturbing user experiences, for example, without significantly interrupting a user's attention or ability to perform a task. In one aspect, a process described herein determines that a user is in a “high stress” state and help the user calm down, e.g., based on detecting physiology corresponding to stressed state, the device may provide relaxing content (e.g., meditation virtual content, relaxing music, etc.).

In some implementations, the feedback mechanism may be selected based on a characteristic of an environment of the user (e.g., real-world physical environment, a virtual environment, or a combination of each). The device (e.g., a handheld, laptop, desktop, or head-mounted device (HMD)) provides an experience (e.g., a visual and/or auditory experience) of the real-world physical environment, an extended reality (XR) environment, or a combination of each (e.g., mixed reality environment) to the user. The device obtains, with one or more sensors, physiological data (e.g., electroencephalography (EEG) amplitude, pupil modulation, eye gaze saccades, heart rate, electrodermal activity/skin conductance, etc.) associated with the user. Based on the obtained physiological data, the techniques described herein can determine a user's stress level 3 (e.g., calm, eustress, distress, etc.) and a type of stress (e.g., physical, cognitive, social, etc.) during the experience (e.g., a learning experience). Based on the physiological data and associated physiological response, the techniques can provide feedback to the user that the current stress level differs from an intended stress level of the experience, recommend similar content or similar portions of the experience, and/or adjust content or feedback mechanisms corresponding to the experience.

Physiological response data, such as EEG amplitude/frequency, pupil modulation, eye gaze saccades, heart rate, electrodermal activity (EDA), etc., can depend on the individual, characteristics of the scene in front of him or her (e.g., video content), attributes of the physical environment surrounding the user including the activity/movement of the user, and the feedback mechanism that is presented therein. Physiological response data can be obtained while using a device with eye tracking technology (and other physiologic sensors) while users perform tasks that demand varying levels of stress, such as interactions to a video (e.g., a high stress video such as walking a plank in XR). In some implementations, physiological response data can be obtained using other sensors, such as EEG sensors or EDA sensors. Observing repeated measures of physiological response data to an experience can give insights about the underlying stress state of the user at different time scales. These metrics of stress can be used to provide feedback during an experience.

Several different experiences can utilize the techniques described herein regarding assessing stress levels. For example, a learning experience could notify a pupil to calm down when he or she appears to be experiencing stress. Another example may be a workplace experience of notifying a worker who needs to take a brief break on his or her current task that is creating high stress environment. For example, providing feedback to a surgeon who may be getting a little tired during a long surgery, alerting a truck driver on a long drive he or she is losing focus and may need to pull over to sleep, and the like. The techniques described herein can be customized to any user and experience that may need some type of feedback mechanism to enter or maintain one or more particular stress levels.

Some implementations assess physiological data and other user information to help improve a user experience. In such processes, user preferences and privacy should be respected, as examples, by ensuring the user understands and consents to the use of user data, understands what types of user data are used, has control over the collection and use of user data and limiting distribution of user data, for example, by ensuring that user data is processed locally on the user's device. Users should have the option to opt in or out with respect to whether their user data is obtained or used or to otherwise turn on and off any features that obtain or use user information. Moreover, each user should have the ability to access and otherwise find out anything that the system has collected or determined about him or her.

In general, one innovative aspect of the subject matter described in this specification can be embodied in methods that include the actions of obtaining physiological data associated with a user during an experience in an environment, determining a context of the experience based on sensor data of the environment, determining a stress level of the user during a portion of the experience based on the obtained physiological data and the context of the experience, and providing a feedback mechanism based on the stress level.

These and other embodiments can each optionally include one or more of the following features.

In some aspects, determining a stress level of the user during the portion of the experience further includes determining a type of stress of the user based on the sensor data, and providing the feedback mechanism during the experience is further based on the type of stress.

In some aspects, the method further includes providing a notification to the user based on the stress level. In some aspects, the method further includes customizing content included in the experience based on the stress level of the user.

In some aspects, the stress level is a first stress level, and the method further includes obtaining, using a sensor, first physiological data associated with a physiological response of the user to the feedback mechanism, and determining a second stress level of the user based on the physiological response of the user to the feedback mechanism.

In some aspects, the method further includes assessing the second stress level of the user based on the physiological response of the user to the feedback mechanism, and determining whether the feedback mechanism reduced stress of the user by comparing the second stress level to the first stress level.

In some aspects, determining the context of the experience includes generating a scene understanding of the environment based on the sensor data of the environment, the scene understanding including visual or auditory attributes of the environment, and determining the context of the experience based on the scene understanding of the environment.

In some aspects, the sensor data includes image data, and generating the scene understanding is based at least on performing semantic segmentation of the image data and detecting one or more objects within the environment based on the semantic segmentation. In some aspects, the sensor data includes location data of the user, and determining the context of the experience includes determining a location of the user within the environment based on the location data.

In some aspects, determining the context of the experience includes determining an activity of the user based on the scene understanding of the environment. In some aspects, determining the context of the experience includes determining an activity of the user based on a user's schedule.

In some aspects, determining the context of the experience includes determining that the user is consuming food. In some aspects, the stress level and the feedback mechanism are determined based on the user consuming food.

In some aspects, the physiological data includes at least one of skin temperature, respiration, photoplethysmogram (PPG), electrodermal activity (EDA), eye gaze tracking, and pupillary movement that is associated with the user.

In some aspects, the stress level is assessed using a statistical or machine learning-based classification technique. In some aspects, the method further includes providing a notification to the user based on the stress level. In some aspects, the method further includes identifying the portion of the experience associated with the stress level. In some aspects, the method further includes customizing content of the experience based on the stress level of the user.

In some aspects, the device is a head-mounted device (HMD) and the environment includes an extended reality environment.

In accordance with some implementations, a non-transitory computer readable storage medium has stored therein instructions that are computer-executable to perform or cause performance of any of the methods described herein. In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein.

### BRIEF DESCRIPTION OF THE DRAWINGS

So that the present disclosure can be understood by those of ordinary skill in the art, a more detailed description may be had by reference to aspects of some illustrative implementations, some of which are shown in the accompanying drawings.

FIG. 1 illustrates a device displaying a visual and/or auditory experience and obtaining physiological data from a user in accordance with some implementations.

FIG. 2 illustrates a pupil of the user of FIG. 1 in which the diameter of the pupil varies with time in accordance with some implementations.

FIG. 3 illustrates detecting a stress level of the user viewing content based on physiological data and context data in accordance with some implementations.

FIG. 4 illustrates a system diagram for detecting a stress level of the user viewing content based on physiological data and context data in accordance with some implementations.

FIG. 5 is a flowchart representation of a method for predicting a stress level of the user viewing content based on physiological data and context data, and providing a feedback mechanism based on the stress level in accordance with some implementations.

FIG. 6 illustrates device components of an exemplary device in accordance with some implementations.

FIG. 7 illustrates an example head-mounted device (HMD) in accordance with some implementations.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DESCRIPTION

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects or variants do not include all of the specific details described herein. Moreover, well-known systems, methods, components, devices and circuits have not been described in exhaustive detail so as not to obscure more pertinent aspects of the example implementations described herein.

FIG. 1 illustrates a real-world environment 5 including a device 10 with a display 15. In some implementations, the device 10 displays content 20 to a user 25, and a visual characteristic 30 that is associated with content 20. For example, content 20 may be a button, a user interface icon, a text box, a graphic, etc. In some implementations, the visual characteristic 30 associated with content 20 includes visual characteristics such as hue, saturation, size, shape, spatial frequency, motion, highlighting, etc. For example, content 20 may be displayed with a visual characteristic 30 of green highlighting covering or surrounding content 20.

In some implementations, content 20 may be a visual experience (e.g., an education experience), and the visual characteristic 30 of the visual experience may continuously change during the visual experience. As used herein, the phrase “experience” refers to a period of time during which a user uses an electronic device and has one or more stress levels. In one example, a user has an experience in which the user perceives a real-world environment while holding, wearing, or being proximate to an electronic device that includes one or more sensors that obtain physiological data that is indicative of the user's stress level. In another example, a user has an experience in which the user perceives content displayed by an electronic device while the same or another electronic obtains physiological data (e.g., pupil data, EEG data, etc.) to assess the user's stress level. In another example, a user has an experience in which the user holds, wears, or is proximate to an electronic device that provides a series of audible or visual instructions that guide the experience. For example, the instructions may instruct the user to have particular stress levels during particular time segments of the experience, e.g., instructing the user to focus on his or her attention to a particular portion of the educational video, etc. During such an experience, the same or another electronic device may obtain physiological data to assess the user's stress level.

In some implementations, the visual characteristic 30 is a feedback mechanism for the user that is specific to the experience (e.g., a visual or audio cue to focus on a particular task during an experience, such as paying attention during a particular part of an education/learning experience). In some implementations, the visual experience (e.g., content 20) can occupy the entire display area of display 15. For example, during an education experience, content 20 may be a cooking video or sequence of images that may include visual and/or audio cues as the visual characteristic 30 presented to the user to pay attention. Other visual experiences that can be displayed for content 20 and visual and/or audio cues for the visual characteristic 30 will be further discussed herein.

The device 10 obtains physiological data (e.g., EEG amplitude/frequency, pupil modulation, eye gaze saccades, etc.) from the user 25 via a sensor 35. For example, the device 10 obtains pupillary data 40 (e.g., eye gaze characteristic data). While this example and other examples discussed herein illustrate a single device 10 in a real-world environment 5, the techniques disclosed herein are applicable to multiple devices and multiple sensors, as well as to other real-world environments/experiences. For example, the functions of device 10 may be performed by multiple devices.

In some implementations, as illustrated in FIG. 1, the device 10 is a handheld electronic device (e.g., a smartphone or a tablet). In some implementations the device 10 is a laptop computer or a desktop computer. In some implementations, the device 10 has a touchpad and, in some implementations, the device 10 has a touch-sensitive display (also known as a “touch screen” or “touch screen display”). In some implementations, the device 10 is a wearable head mounted display (HMD).

In some implementations, the device 10 includes an eye tracking system for detecting eye position and eye movements. For example, an eye tracking system may include one or more infrared (IR) light-emitting diodes (LEDs), an eye tracking camera (e.g., near-IR (NIR) camera), and an illumination source (e.g., an NIR light source) that emits light (e.g., NIR light) towards the eyes of the user 25. Moreover, the illumination source of the device 10 may emit NIR light to illuminate the eyes of the user 25 and the NIR camera may capture images of the eyes of the user 25. In some implementations, images captured by the eye tracking system may be analyzed to detect position and movements of the eyes of the user 25, or to detect other information about the eyes such as pupil dilation or pupil diameter. Moreover, the point of gaze estimated from the eye tracking images may enable gaze-based interaction with content shown on the near-eye display of the device 10.

In some implementations, the device 10 has a graphical user interface (GUI), one or more processors, memory and one or more modules, programs or sets of instructions stored in the memory for performing multiple functions. In some implementations, the user 25 interacts with the GUI through finger contacts and gestures on the touch-sensitive surface. In some implementations, the functions include image editing, drawing, presenting, word processing, website creating, disk authoring, spreadsheet making, game playing, telephoning, video conferencing, e-mailing, instant messaging, workout support, digital photographing, digital videoing, web browsing, digital music playing, and/or digital video playing. Executable instructions for performing these functions may be included in a computer readable storage medium or other computer program product configured for execution by one or more processors.

In some implementations, the device 10 employs various physiological sensor, detection, or measurement systems. Detected physiological data may include, but is not limited to, EEG, electrocardiography (ECG), electromyography (EMG), functional near infrared spectroscopy signal (fNIRS), blood pressure, skin conductance, or pupillary response. The device 10 maybe communicatively coupled to an additional sensor. For example, sensor 17 (e.g., an EDA sensor) maybe communicatively coupled to device 10 via a wired or wireless connection, and sensor 17 may be located on the skin of the user 25 (e.g., on the arm as illustrated, or placed on the hand/fingers of the user). For example, sensor 17 can be utilized for detecting EDA (e.g., skin conductance), heart rate, or other physiological data that utilizes contact with the skin of a user. Moreover, the device 10 (using one or more sensors) may simultaneously detect multiple forms of physiological data in order to benefit from synchronous acquisition of physiological data. Moreover, in some implementations, the physiological data represents involuntary data, e.g., responses that are not under conscious control. For example, a pupillary response may represent an involuntary movement.

In some implementations, one or both eyes 45 of the user 25, including one or both pupils 50 of the user 25 present physiological data in the form of a pupillary response (e.g., pupillary data 40). The pupillary response of the user 25 results in a varying of the size or diameter of the pupil 50, via the optic and oculomotor cranial nerve. For example, the pupillary response may include a constriction response (miosis), e.g., a narrowing of the pupil, or a dilation response (mydriasis), e.g., a widening of the pupil. In some implementations, the device 10 may detect patterns of physiological data representing a time-varying pupil diameter.

In some implementations, a pupillary response may be in response to an auditory feedback that one or both ears 60 of the user 25 detect (e.g., an audio notification to the user). For example, device 10 may include a speaker 12 that projects sound via sound waves 14. The device 10 may include other audio sources such as a headphone jack for headphones, a wireless connection to an external speaker, and the like.

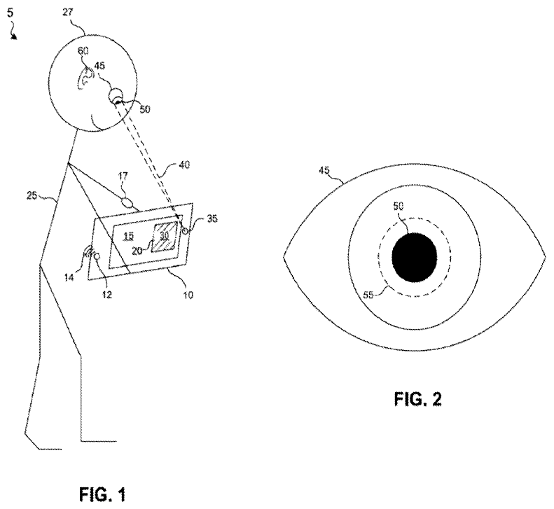

FIG. 2 illustrates a pupil 50 of the user 25 of FIG. 1 in which the diameter of the pupil 50 varies with time. Pupil diameter tracking may be potentially indicative of a physiological state of a user. As shown in FIG. 2, a present physiological state (e.g., present pupil diameter) may vary in contrast to a past physiological state (e.g., past pupil diameter 55). For example, the present physiological state may include a present pupil diameter and a past physiological state may include a past pupil diameter.

The physiological data may vary in time and the device 10 may use the physiological data to measure one or both of a user's physiological response to the visual characteristic 30 or the user's intention to interact with content 20. For example, when presented with content 20, such as a list of content experiences (e.g., meditation environments), by a device 10, the user 25 may select an experience without requiring the user 25 to complete a physical button press. In some implementations, the physiological data may include the physiological response of a visual or an auditory stimulus of a radius of the pupil 50 after the user 25 glances at content 20, measured via eye-tracking technology (e.g., via a HMD). In some implementations, the physiological data includes EEG amplitude/frequency data measured via EEG technology, or EMG data measured from EMG sensors or motion sensors.

Returning to FIG. 1, a physical environment refers to a physical world that people can sense and/or interact with without aid of electronic devices. The physical environment may include physical features such as a physical surface or a physical object. For example, the physical environment corresponds to a physical park that includes physical trees, physical buildings, and physical people. People can directly sense and/or interact with the physical environment such as through sight, touch, hearing, taste, and smell. In contrast, an extended reality (XR) environment refers to a wholly or partially simulated environment that people sense and/or interact with via an electronic device. For example, the XR environment may include augmented reality (AR) content, mixed reality (MR) content, virtual reality (VR) content, and/or the like. With an XR system, a subset of a person's physical motions, or representations thereof, are tracked, and, in response, one or more characteristics of one or more virtual objects simulated in the XR environment are adjusted in a manner that comports with at least one law of physics. As one example, the XR system may detect head movement and, in response, adjust graphical content and an acoustic field presented to the person in a manner similar to how such views and sounds would change in a physical environment. As another example, the XR system may detect movement of the electronic device presenting the XR environment (e.g., a mobile phone, a tablet, a laptop, or the like) and, in response, adjust graphical content and an acoustic field presented to the person in a manner similar to how such views and sounds would change in a physical environment. In some situations (e.g., for accessibility reasons), the XR system may adjust characteristic(s) of graphical content in the XR environment in response to representations of physical motions (e.g., vocal commands).

There are many different types of electronic systems that enable a person to sense and/or interact with various XR environments. Examples include head mountable systems, projection-based systems, heads-up displays (HUDs), vehicle windshields having integrated display capability, windows having integrated display capability, displays formed as lenses designed to be placed on a person's eyes (e.g., similar to contact lenses), headphones/earphones, speaker arrays, input systems (e.g., wearable or handheld controllers with or without haptic feedback), smartphones, tablets, and desktop/laptop computers. A head mountable system may have one or more speaker(s) and an integrated opaque display. Alternatively, a head mountable system may be configured to accept an external opaque display (e.g., a smartphone). The head mountable system may incorporate one or more imaging sensors to capture images or video of the physical environment, and/or one or more microphones to capture audio of the physical environment. Rather than an opaque display, a head mountable system may have a transparent or translucent display. The transparent or translucent display may have a medium through which light representative of images is directed to a person's eyes. The display may utilize digital light projection, OLEDs, LEDs, uLEDs, liquid crystal on silicon, laser scanning light source, or any combination of these technologies. The medium may be an optical waveguide, a hologram medium, an optical combiner, an optical reflector, or any combination thereof. In some implementations, the transparent or translucent display may be configured to become opaque selectively. Projection-based systems may employ retinal projection technology that projects graphical images onto a person's retina. Projection systems also may be configured to project virtual objects into the physical environment, for example, as a hologram or on a physical surface.

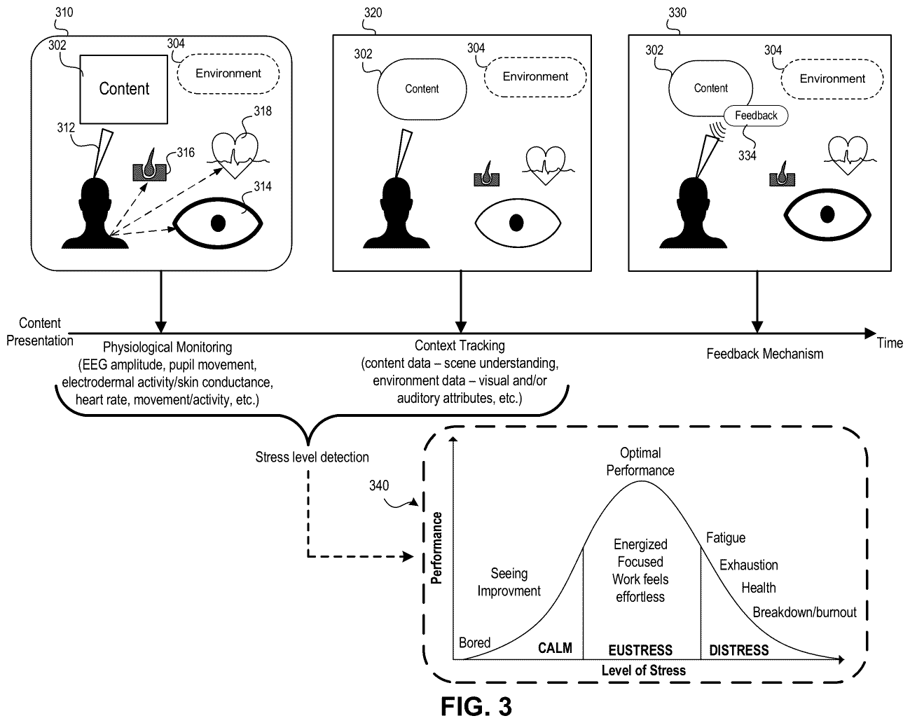

FIG. 3 illustrates detecting a stress level of the user viewing content based on physiological data and context data. In particular, FIG. 3 illustrates a user (e.g., user 25 of FIG. 1) being presented with content 302 in an environment 304 during a content presentation where the user, via obtained physiological data, has a physiological response to the content (e.g., the user looks towards portions of the content as detected by eye gaze characteristic data). For example, at content presentation instant 310, a user is being presented with content 302 that includes visual content (e.g., a video), and the user's physiologic data such as eye gaze characteristic data 312, pupillary data 314, EDA data 316, and heart rate data 318 is monitored as a baseline. Then, at content presentation instant 320, the content 302 and environment 304 is being analyzed by a context analysis instruction set to determine context data for the experience of the user (e.g., the experience of being present in the current physical environment while watching video content on an electronic device such as an HMD). Determining context data of the experience may involve using computer vision to generate a scene understanding of the visual and/or auditory attributes of the physical environment (e.g., environment 304), such as where is the user, what is the user doing, what objects are nearby. Additionally, or alternatively, determining context data of the experience may involve determining a scene understanding of the visual and/or auditory attributes of the content presentation (e.g., content 302, such as a video). For example, content 302 and environment 304 may include one or more people, objects, or other background objects that are within view of the user that may be detected by an objection detection algorithm, face detection algorithm, or the like.

After a segment of time after the user's physiological data is analyzed (e.g., by a physiological data instruction set) and the context data of the content 302 and/or environment 304 is analyzed (e.g., by a context instruction set), content presentation instant 330 is presented to the user with a feedback mechanism 334 because the stress level assessment was that the user was may have exhibited higher stress levels than desired. For example, a user is exhibiting a high stress level while at work and the feedback mechanism indicates as such to the user and may provide the user with some alternatives for calming (e.g., meditation music, a relaxing XR environment, etc.). As illustrated, the stress level graph 340 provides a possible use case of comparing the detected level of stress of the user and the performance level associated with that stress. For example, for the above example of exhibiting a high stress level (e.g., a “distress” level), then the feedback mechanism could alert the user of this high level to try and calm them to a reasonable level of stress (e.g., a “eustress” level) where an “optimal performance” may be associated with those levels. Additionally, it is also known that low levels stress (e.g., a “calm” level) may be associated with lower levels of performance because the user may be bored. Thus, in a workplace environment, the processes described herein may detect this low level of stress, and provide a feedback mechanism to alert the user of this current performance metric and offer suggestions on ways to increase the stress level, and thus in turn, increase performance (e.g., tell the user to take a break and exercise.) The user's stress level assessment can be continuously monitored throughout the presentation of the content 302.

The feedback mechanism 334 may include a visual presentation. For example, an icon may appear, or a text box may appear instructing the user to pay attention. In some implementations, the feedback mechanism 334 may include an auditory stimulus. For example, spatialized audio may be presented to redirect the user's attention towards the particular areas of the content presentation (e.g., if determined the user was exhibiting high stress, could steer the user's attention towards something relaxing in content). In some implementations, the feedback mechanism 334 may include an entire display of visual content (e.g., a relaxation video over the entire display of the device). Alternatively, the feedback mechanism 334 may include visual content around the frame of the display of the device (e.g., on a mobile device, a virtual frame of the display be created to acquire the user's attention from a particular stress level). In some implementations, the feedback mechanism 334 may include a combination of visual content (e.g., a notification window, an icon, or other visual content described herein) and/or an auditory stimulus. For example, a notification window or arrow may direct the user to a particular content area and an audio signal may be presented that directs the user. These visual and/or auditory cues can help direct the user to particular feedback mechanisms that can aid the user in coping with different stress levels to increase his or her performance levels (for a work experience), or simply for comfortable viewing of the content 302 (e.g., providing meditation if determined the user is in a stressful environment or situation).

In some implementations, the feedback mechanism 334 may be utilized for stress eating detection and feedback (e.g., understanding a user's state (stress) to help a user). For example, a stress level and the feedback mechanism 334 may be determined based on the user consuming food as determined in the context of the environment (e.g., images of a particular food or the user eating a particular food). For example, stress detection and context awareness of what the user is eating or about to eat (e.g., a bag of potato chips), useful feedback might be provided to a user (e.g., to meditate or relieve stress in a healthier way). In particular, the electronic device 10 may employ an object detection/classification unhealthy food algorithm based on acquired images of the user and/or the user's environment. Thus, when the user is or is about to consume a food (or an amount of food) that is classified as unhealthy, a user may be provided with a notification (visual and/or audible) to encourage the user to find a healthier way to cope with detected stress.

Over time a user's stress and food consumption may be tracked to identify correlations and information that can be provided to a user to help the user understand his or her behavior and/or habits. Feedback provided to the user may enable a user to understand, appreciate, and recognize his or her habits and mechanisms for coping with stress and/or to change such habits over time. Feedback provided to a user may encourage the user to substitute healthy stress relief alternatives for unhealthy food consumption behaviors and habits. In some implementations, the system accounts for a user's preferences, goals, and/or history in providing feedback. For example, based on a user identified goals of improving fitness, feedback to the user may encourage a user to exercise or meditate rather than consuming unhealthy food during stressful times.

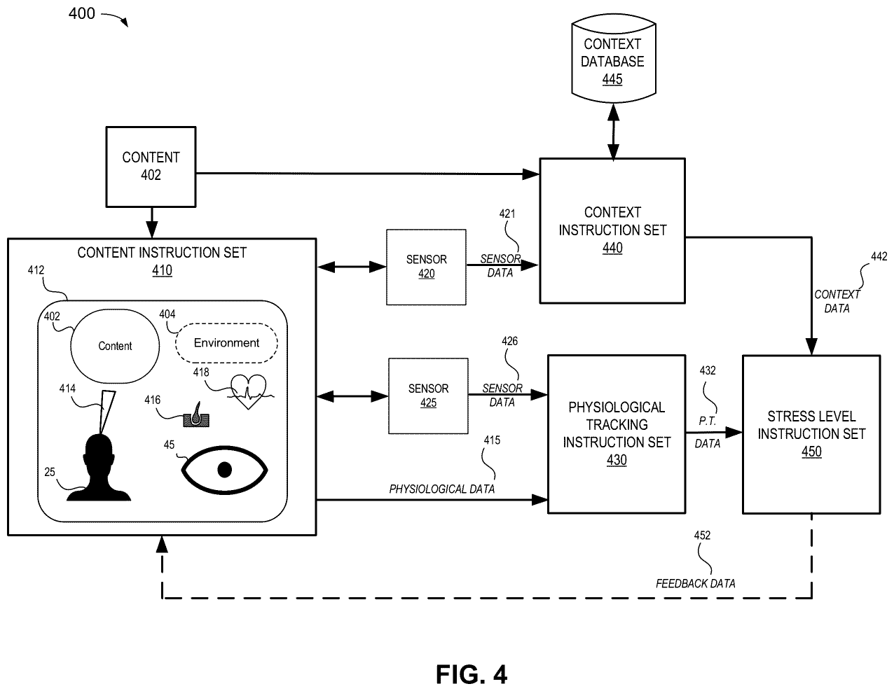

FIG. 4 is a system flow diagram of an example environment 400 in which a stress level assessment system can assess a stress level of a user based on physiological data and context data, and provide a feedback mechanism(s) within the presentation of the content according to some implementations. In some implementations, the system flow of the example environment 400 is performed on a device (e.g., device 10 of FIG. 1), such as a mobile device, desktop, laptop, or server device. The content of the example environment 400 can be displayed on a device (e.g., device 10 of FIG. 1) that has a screen (e.g., display 15) for displaying images and/or a screen for viewing stereoscopic images such as a HMD. In some implementations, the system flow of the example environment 400 is performed on processing logic, including hardware, firmware, software, or a combination thereof. In some implementations, the system flow of the example environment 400 is performed on a processor executing code stored in a non-transitory computer-readable medium (e.g., a memory).

The system flow of the example environment 400 acquires and presents content (e.g., video content or a series of image data) to a user, analyzes the content and/or the environment for context data, obtains physiological data associated with the user during presentation of the content, assesses a stress level of the user based on the physiological data of the user and the context data, and provides a feedback mechanism based on the stress level (e.g., a notification/alert based on a high stress threshold and/or a low stress threshold). For example, a stress level assessment technique described herein determines, based on obtained physiological data, a user's stress level during an experience (e.g., watching a video) by providing a feedback mechanism that is based on the stress level of the user (e.g., a notification, auditory signal, an alert, an icon, and the like, that alerts the user that they may be at a particular stress level during the presentation of content).

The example environment 400 includes a content instruction set 410 that is configured with instructions executable by a processor to provide and/or track content 402 for display on a device (e.g., device 10 of FIG. 1). For example, the content instruction set 410 provides content presentation instant 412 that includes content 402 to a user 25 while user is within a physical environment 404 (e.g., a room, outside, etc.). For example, content 402 may include background image(s) and sound data (e.g., a video). The content presentation instant 412 could be an XR experience (e.g., an education experience), or content presentation instant 412 could be a MR experience that includes some CGR content and some images of a physical environment. Alternatively, the user could be wearing a HMD and is looking at a real physical environment either via a live camera view, or the HMD allows a user to look through the display, such as wearing smart glasses that user can see through, but still be presented visual and/or audio cues. During an experience, while a user 25 is viewing the content 402, pupillary data 414 (e.g., pupillary data 40 such as eye gaze characteristic data) of the user's eyes can be monitored and sent as physiological data 415. Additionally, other physiological data can be monitored and sent as physiological data 415 such as EDA data 416 and heart rate data 418.

The environment 400 further includes a physiological tracking instruction set 430 to track a user's physiological attributes as physiological tracking data 432 using one or more of the techniques discussed herein or as otherwise may be appropriate. For example, the physiological tracking instruction set 430 may acquire physiological data 415 (e.g., pupillary data 414) from the user 25 viewing the content 402. Additionally, or alternatively, a user 25 may be wearing a sensor 425 (e.g., sensor 17 of FIG. 1, such as an EEG sensor, an EDA sensor, heart rate sensor, etc.) that generates sensor data 426 (e.g. EEG data, EDA data 416, heart rate data 418) as additional physiological data. Thus, as the content 402 is presented to the user as content presentation instant 412, the physiological data 415 (e.g., pupillary data 414) and/or sensor data 426 is sent to the physiological tracking instruction set 430 to track a user's physiological attributes as physiological tracking data 432, using one or more of the techniques discussed herein or as otherwise may be appropriate.

In an example implementation, the environment 400 further includes a context instruction set 440 that is configured with instructions executable by a processor to obtain the experience data presented to the user (e.g., content 402) and other sensor data (e.g., image data of the environment 404, the user's 25 face and/or eye's, etc.), and generate context data 442 (e.g., identifying people, objects, etc. of the content 402 and the environment 404). For example, the context instruction set 440 acquires content 402 and sensor data 421 (e.g., image data) from the sensor 420 (e.g., an RGB camera, a depth camera, etc.) and determines context data 442 based on identifying areas of the content while the user is viewing the presentation of the content 402 (e.g., a first time viewed content/video). Alternatively, the context instruction set 440 selects context data associated with content 402 from a context database 445 (e.g., if the content 402 was previously analyzed by the context instruction set, i.e., a previously viewed/analyzed video). In some implementations, the context instruction set 440 generates a scene understanding associated with content 402 and/or environment 404 as the context data 442. For example, the scene understanding can be utilized to track the overall context of what the user may be focused on during the presentation of content 402, or where the user is, what the user is doing, what physical objects or people are in the vicinity of the user with respect to the environment 404.

In an example implementation, the environment 400 further includes a stress level instruction set 450 that is configured with instructions executable by a processor to assess the stress level of a user based on a physiological response (e.g., eye gaze response) using one or more of the techniques discussed herein or as otherwise may be appropriate. For example, the stress level be assessed such as determining where the user's stress level may be with respect to an indicator, such as stress level graph 340 of FIG. 3. In particular, the stress level instruction set 450 acquires physiological tracking data 432 from the physiological tracking instruction set 430 and context data 442 from the context instruction set 440 (e.g., scene understanding data) and determines the stress level of the user 25 during the presentation of the content 402 and based on attributes of the physical environment 404 that the user is watching the content 402. For example, the context data 442 may provide a scene analysis that can be used by the stress level instruction set 450 to understand what the person is looking at, where they are at, etc., and improve the determination of the stress level. In some implementations, the stress level instruction set 450 can then provide feedback data 452 (e.g., visual and/or audible cues) to the content instruction set 410 based on the stress level assessment. For example, finding defined markers of high/low levels of stress and providing performance feedback during an education experience could enhance a user's learning experience, provide additional benefits from the education session, and provide a guided and supportive teaching approach (e.g., a scaffolding teaching method) for users to advance through their education practice.

In some implementations, the feedback data 452 could be utilized by the content instruction set 410 to present an audio and/or visual feedback cue or mechanism to the user 25 to relax and focus on breathing during the high-level stress situation (e.g., over anxious about an upcoming test). In an educational experience, the feedback cue to the user could be a gentle reminder (e.g., a soothing or calming visual and/or audio alarm) to get back on task of studying, based on the assessment from the stress level instruction set 450 that the user 25 is mind wandering because the user 25 was bored (e.g., a low level stress indication).

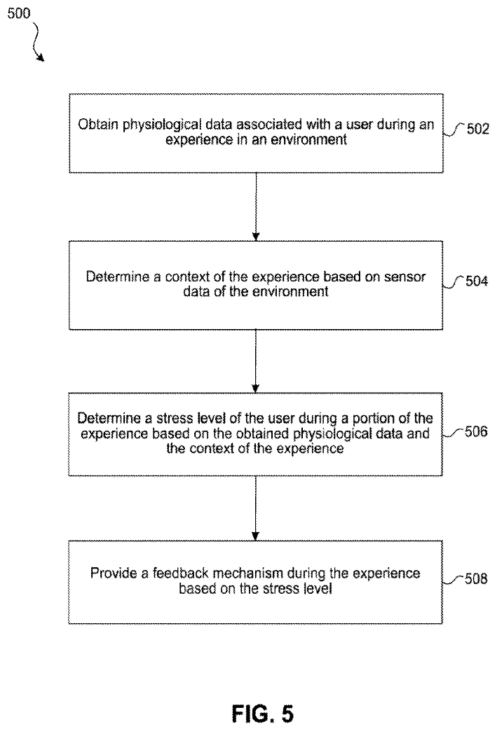

FIG. 5 is a flowchart illustrating an exemplary method 500. In some implementations, a device such as device 10 (FIG. 1) performs the techniques of method 500 to assess a stress level of the user viewing content based on physiological data and context data, and providing a feedback mechanism based on the detected stress level. In some implementations, the techniques of method 500 are performed on a mobile device, desktop, laptop, HMD, or server device. In some implementations, the method 500 is performed on processing logic, including hardware, firmware, software, or a combination thereof. In some implementations, the method 500 is performed on a processor executing code stored in a non-transitory computer-readable medium (e.g., a memory).

At block 502, the method 500 obtains physiological data (e.g., EEG amplitude/frequency, pupil modulation, eye gaze saccades, EDA, heart rate, etc.) associated with a user during an experience in an environment. For example, obtaining the physiological data may involve obtaining images of the eye or EOG data from which gaze direction/movement can be determined. Obtaining the physiological data may involve obtaining images of the eye or EOG data from which gaze direction/movement can be determined, electrodermal activity/skin conductance, heart rate, via sensors on a watch. Additionally, facial recognition via HMD may be included as physiological data (e.g., reconstruction of the user's face).

In some implementations, obtaining the physiological data associated with a physiological response of the user includes monitoring for a response or lack of response occurring within a predetermined time following the presenting of the content or a user performing a task. For example, the system may wait for up to five seconds after an event within the video to see if a user looks in a particular direction (e.g., a physiological response).

In some implementations, obtaining physiological data (e.g., pupillary data 40) is associated with a gaze of a user that may involve obtaining images of the eye or electrooculography signal (EOG) data from which gaze direction and/or movement can be determined. In some implementations, the physiological data includes at least one of skin temperature, respiration, photoplethysmogram (PPG), electrodermal activity (EDA), eye gaze tracking, and pupillary movement that is associated with the user.

Some implementations obtain physiological data and other user information to help improve a user experience. In such processes, user preferences and privacy should be respected, as examples, by ensuring the user understands and consents to the use of user data, understands what types of user data are used, has control over the collection and use of user data and limiting distribution of user data, for example, by ensuring that user data is processed locally on the user's device. Users should have the option to opt in or out with respect to whether their user data is obtained or used or to otherwise turn on and off any features that obtain or use user information. Moreover, each user will have the ability to access and otherwise find out anything that the system has collected or determined about him or her. User data is stored securely on the user's device. User data that is used as input to a machine learning model is stored securely on the user's device, for example, to ensure the user's privacy. The user's device may have a secure storage area, e.g., a secure enclave, for securing certain user information, e.g., data from image and other sensors that is used for face identification, face identification, or biometric identification. The user data associated with the user's body and/or attentive state may be stored in such a secure enclave, restricting access to the user data and restricting transmission of the user data to other devices to ensure that user data is kept securely on the user's device. User data may be prohibited from leaving the user's device and may be used only in machine learning models and other processes on the user's device.

At block 504, the method 500 determines a context of the experience based on sensor data of the environment. For example, determining a context may involve using computer vision to generate a scene understanding of the visual and/or auditory attributes of the environment—where is the user, what is the user doing, what objects are nearby. Additionally, a scene understanding of the content presented to the user could be generated that includes the visual and/or auditory attributes of what the user was watching.

In some aspects, different contexts of the content presented and the environment are analyzed to determine where the user is, what the user is doing, what objects or people are nearby in the environment or within the content, what the user did earlier (e.g., meditated in the morning). Additionally, context analysis may include image analysis (semantic segmentation), audio analysis (jarring sounds), location sensors (where user is), motion sensors (fast moving vehicle), and even access other user data (e.g., a user's calendar). In an exemplary implementation, the method 500 may further include determining the context of the experience by generating a scene understanding of the environment based on the sensor data of the environment, the scene understanding including visual or auditory attributes of the environment, and determining the context of the experience based on the scene understanding of the environment.

In some implementations, the sensor data includes image data, and generating the scene understanding is based at least on performing semantic segmentation of the image data and detecting one or more objects within the environment based on the semantic segmentation. In some implementations, determining the context of the experience includes determining an activity of the user based on the scene understanding of the environment. In some implementations, the sensor data includes location data of the user, and determining the context of the experience includes determining a location of the user within the environment based on the location data.

In some implementations, determining the context of the experience may involve identifying an object or individual with which the user is interacting. Determining the context of the experience may involve determining that the user is conversing with another individual. Determining the context of the experience may involve determining that an interaction or conversation with another individual is likely (or unlikely) to evoke a stressful state in the user. Assessing whether an individual is more or less likely to evoke a stressful response for the user may involve identifying the individual, and classifying the individual based on appearance of the individual, based on an action of the individual, and/or based on an activity that the individual is engaged in. For example, if the other individual is identified as the user's boss at work, the boss can be identified via facial recognition, or be classified as a supervising coworker. The stress of the user can then be tracked based on his or her stress levels when interacting with that individual that was classified as his or her boss. Providing feedback to a user (or his or her therapist) regarding the user's higher stress levels when interacting with his or her boss, may be useful when evaluating stress therapy techniques to better cope with high stress situations.

In some implementations, determining the context of the experience may involve determining a scene understanding or scene knowledge that a particular location (e.g., a particular room, building, etc.) that a user experiences is more or less likely to lead to a stressful state (e.g., based on past stressful experiences that occurred there). Determining a scene understanding or scene knowledge of an experience may involve monitoring low level characteristics of a scene that can evoke stress. For example, loud noises, looming sounds, bright flashes of light, sirens, rumbling sounds, and the like, may be monitored and analyzed as part of the scene understanding or scene knowledge. Additionally, scene knowledge may provide information that a particular activity or content might be troubling or stressful. For example, scene knowledge may include experiences or events that the user is currently participating in such as interviewing, reading a disturbing news story, watching a scary movie, playing a violent video game, and the like. Understanding scene knowledge may involve other stressful experiences such as threatening stimuli (e.g., an aggressive dog), harm done to a loved one, perceived physical danger to the user (e.g., an oncoming car), online bullying, being berated in person, and the like. Determining the context of an experience may involve determining a user activity and/or a type of the environment based on the scene understanding of the environment.

In some implementations, determining the context of the experience includes determining an activity of the user based on a user's schedule. For example, the system may access a user's calendar to determine if a particular event is occurring when the particular stress level is assessed (e.g., the user is late for an important meeting or class, or is scheduled to present in front of a group in the near future).

At block 506, the method 500 determines a stress level of the user during a portion of the experience based on the obtained physiological data and the context of the experience. For example, a machine learning model may be used to determine stress levels and/or type of stress (e.g., physical, cognitive, social, etc.) based on eye tracking and other physiological data, and audio/visual content of the experience and/or the environment. For example, one or more physiological characteristics may be determined, aggregated, and used to classify the user's stress level using statistical or machine learning techniques. In some implementations, the response may be compared with the user's own prior responses or typical user stress levels to similar content of a similar experience and/or similar environment attributes.

In some implementations, determining that the user has a particular threshold of stress (e.g., high, low, etc.) includes determining a level of stress as a sliding scale. For example, the system could determine a level of stress as a stress barometer that can be customized based on the type of content shown during the user experience. If a high level of stress, if for education, a content developer can design an environment for the experience that will provide the user the “best” environment for a learning experience. For example, tune the ambience lighting so the user can be at the optimal levels to learn during the experience.

In some implementations, stress level may be determined using statistical or machine learning-based classification techniques. For example, determining that the user has a stress level includes using a machine learning model trained using ground truth data that includes self-assessments in which users labelled portions of experiences with stress level labels. For example, to determine the ground truth data that includes self-assessments, a group of subjects, while watching a cooking instructional video, could be prompted at different time intervals (e.g., every 30 seconds) Alternatively, or additionally, the ground truth data that includes self-assessments while watching a video includes different examples stress events. For example, the different stress events could be displayed and transitioned between each stress event to each subject in an XR environment while a user is wearing an HMD. The “stress events” may include high stress event such as walking on a virtual plank on a high rise (e.g., physical stress event), having the user take a math test (e.g., cognitive stress event), or simulate a user having to make a presentation to a group of people (e.g., social stress event). Additionally, lower stress events may also be included for recording low stress levels, either in between the high stress events, or shown separately (e.g., a mediation video with calming sounds/music). After each “stress event”, each subject could be prompted at or after a particular stress event in the video content to enter his or her stress level.

In some implementations, one or more pupillary or EEG characteristics may be determined, aggregated, and used to classify the user's stress level using statistical or machine learning techniques. In some implementations, the physiological data is classified based on comparing the variability of the physiological data to a threshold. For example, if the baseline for a user's EEG data is determined during an initial segment of time (e.g., 30-60 seconds), and during a subsequent segment of time following an auditory stimulus (e.g., 5 seconds) the EEG data deviates more than +/−10% from the EEG baseline during the subsequent segment of time, than the techniques described herein could classify the user as transitioned away from the high stress level and entered a second lower stress level. Similarly, the heart rate data and/or EDA data may also be classified based on comparing the variability of the heart rate data and/or EDA data to a particular threshold.

In some implementations, the machine learning model is a neural network (e.g., an artificial neural network), decision tree, support vector machine, Bayesian network, or the like. These labels may be collected from the user beforehand, or from a population of people beforehand, and fine-tuned later on individual users. Creating this labeled data may require many users going through an experience (e.g., a meditation experience) where the users listen to natural sounds with intermixed natural-probes (e.g., an auditory stimulus) and then randomly are asked how focused or relaxed they were (e.g., stress level) shortly after a probe was presented. The answers to these questions can generate a label for the time prior to the question and a deep neural network or deep long short term memory (LSTM) network might learn a combination of features specific to that user or task given those labels (e.g., low stress level, high stress level, etc.).

In some implementations, a context analysis may be obtained or generated, to determine what content the user is focusing on that is creating an increase (or decrease) in stress level (e.g., a person during a social interaction), which may include a scene understanding of the content and/or the physical environment. In an exemplary implementation, the method 500 may further include identifying the portion of the experience associated with the stress level. For example, identifying a portion of the experience associated with a particularly high stress level (e.g., over a high stress threshold), the data may provide a recommendation (or dissuasion) of similar content or portions of the content to the user or to help the content developer improve the content for future users. For example, maybe the goal of the content developer is to increase stress in a video game, decrease stress for a meditation experience, or increase stress if a user is “bored” while studying or at work (e.g., to improve cognitive performance levels).

At block 508, the method 500 provides a feedback mechanism during the experience based on the stress level. The determined stress level could be used to provide feedback to the user via the feedback mechanism which may aid the user, provide statistics to the user, and/or help content creators improve the content of the experience.

In some implementations, predicting a type of stress (e.g., physical, cognitive, social) may be utilized by the processes described herein. In an exemplary implementation, determining a stress level of the user during the portion of the experience further includes determining a type of stress of the user based on the sensor data, and providing the feedback mechanism during the experience is further based on the type of stress. For example, if the user is experiencing a high level stress and the type of stress was determined to be a cognitive stress (e.g., studying for a test), then the feedback mechanism may include video and/or auditory content (e.g., a notification to “take a deep breath”, or adding relaxing music) that can aid the user in lowering his or her stress level to find a better level stress (e.g., higher performance level of studying).

In some implementations, feedback can be provided to a user based on determining that the stress level (e.g., playing an intense video game) differs from an intended stress level of the experience (e.g., content developer wants to increase the stress level for a particular portion of the video game). In some implementations, the method 500 may further include presenting feedback (e.g., audio feedback such as “control your breathing”, visual feedback, etc.) during the experience in response to determining that the stress level differs from a second stress level intended for the experience. In one example, during a portion of an educational experience in which a user is studying for a difficult test, the method determines to present feedback directing the user to focus on breathing based on detecting that the user is instead in a high stress level while studying.

In some implementations, the methods described herein may be implemented for stress eating detection and feedback (e.g., understanding a user's state (stress) to help a user). In an exemplary implementation of method 500, determining the context of the experience includes determining that the user is consuming food, a particular type of food, and/or a quantity of food. Additionally, the stress level and the feedback mechanism may be determined based on the user consuming food. For example, stress detection and context awareness of what the user is eating or about to eat (e.g., a bag of potato chips), useful feedback might be provided to a user (e.g., to meditate or relieve stress in a healthier way). In particular, the electronic device 10 may employ an object detection/classification unhealthy food algorithm (e.g., via the stress level instruction set 450 of FIG. 4) based on acquired images of the user and/or the user's environment. Thus, when the user is or is about to consume a type of food or an amount of food that is classified as unhealthy, a user may be provided with a notification (visual and/or audible) to encourage the user to find a healthier way to cope with detected stress.

In some implementations, determining the context of the experience involves identifying an attribute (e.g., persons, events, characteristics, etc.) of the environment separate from the experience, for example, that may have an impact on the user's stress level. Determining the context of the experience may involve identifying an object and/or determining that an object in the environment is proximate the user, e.g., within a threshold distance of the user and thus more likely to be causing or otherwise impacting the user's stress level. Determining the context of the experience may involve determining that a stimulus in the environment is associated with the stress level of the user, where the stimulus is separate from experience. For example, this may involve determining that a dog barking in the user's house is increasing the user's stress level.

In some implementations, the stress level is a first stress level, and the method further includes obtaining, using a sensor, first physiological data (e.g., EEG amplitude, pupil movement, etc.) associated with a physiological response (or lack of response) of the user to the feedback mechanism, and determining a second stress level of the user based on the physiological response of the user to the feedback mechanism. In some implementations, the method further includes assessing the second stress level of the user based on the physiological response of the user to the feedback mechanism, and determining whether the feedback mechanism reduced stress of the user by comparing the second stress level to the first stress level. For example, the stress levels may be compared with the user's own prior responses or typical user responses to similar stimuli. The stress level may be determined using statistical or machine learning-based classification techniques. Additionally, the determined stress levels could be used to provide feedback to the user/reorient the user, provide statistics to the user, or help content creators; use cases include meditation, learning, breathing, workday.

In some implementations, providing the feedback mechanism includes providing a graphical indicator or sound configured to change the stress level to a second stress level corresponding to stress exhibited by the user (e.g., as detected by the physiological data and/or context data) in the task during the portion of the experience. In some implementations, providing the feedback mechanism includes providing a mechanism for rewinding or providing a break from content associated with the task (e.g., rewinding during a cooking video to replay the last step(s), or pausing an educational lecture for a study break). In some implementations, providing the feedback mechanism includes suggesting a time for another experience based on stress level.

In some implementations, the method 500 further includes adjusting content corresponding to the experience based on the stress level (e.g., customized to the stress level of the user). For example, content recommendation for a content developer can be provided based on determining stress levels during the presented experience and changes of the experience or content presented therein. For example, the user may focus well when particular types of content are provided. In some implementations, the method 500 may further include identifying content based on similarity of the content to the experience, and providing a recommendation of the content to the user based on determining that the user has the stress level during the experience (e.g., mind wandering). In some implementations, the method 500 may further include customizing content included in the experience based on the stress level of the user (e.g., breaking the content into smaller pieces).

In some implementations, content for the experience can be adjusted corresponding to the experience based on the stress level differing from an intended stress level for the experience. For example, content may be adjusted by an experience developer to improve recorded content for a subsequent use for the user or other users. In some implementations, the method 500 may further include adjusting content corresponding to the experience in response to determining that the stress level differs from a second stress level intended for the experience.

In some implementations, the techniques described herein obtain physiological data (e.g., pupillary data 40, EEG amplitude/frequency data, pupil modulation, eye gaze saccades, heart rate data, EDA data, etc.) from the user based on identifying typical interactions of the user with the experience. For example, the techniques may determine that a variability of an eye gaze characteristic of the user correlates with an interaction with the experience. Additionally, the techniques described herein may then adjust a visual characteristic of the experience, or adjust/change a sound associated with the feedback mechanism, to enhance physiological response data associated with future interactions with the experience and/or the feedback mechanism presented within the experience. Moreover, in some implementations, changing a feedback mechanism after the user interacts with the experience informs the physiological response of the user in subsequent interactions with the experience or a particular segment of the experience. For example, the user may present an anticipatory physiological response associated with the change within the experience. Thus, in some implementations, the technique identifies an intent of the user to interact with the experience based on an anticipatory physiological response. For example, the technique may adapt or train an instruction set by capturing or storing physiological data of the user based on the interaction of the user with the experience, and may detect a future intention of the user to interact with the experience by identifying a physiological response of the user in anticipation of the presentation of the enhanced/updated experience.

In some implementations, an estimator or statistical learning method is used to better understand or make predictions about the physiological data (e.g., pupillary data characteristics, EEG data, EDA data, heart rate data, etc.). For example, statistics for EEG data may be estimated by sampling a dataset with replacement data (e.g., a bootstrap method).

In some implementations, the techniques could be trained on many sets of user physiological data and then adapted to each user individually. For example, content creators can customize an education experience (e.g., an instructional cooking video) based on the user physiological data, such as a user may require background music, different ambient lighting for learning, or require more or less audio or visual cues to continue to maintain meditation.

In some implementations, customization of the experience could be controlled by the user. For example, a user could select the experience he or she desires, such as he or she can choose the ambience, background scene, music, etc. Additionally, the user could alter the threshold of providing the feedback mechanism. For example, the user can customize the sensitivity of triggering the feedback mechanism based on prior experience of a session. For example, a user may desire to not have as many feedback notifications and allow some mind wandering (e.g., eye position deviations) before a notification is triggered. Thus, particular experiences can be customized on triggering a threshold when higher criteria is met. For example, some experiences, such as an education experience, a user may not want to be bothered during a study session if he or she is briefly staring off task or mind wandering by briefly looking towards a different area for a moment (e.g., less than 30 seconds) to contemplate what he or she just read. However, the student/reader would want to be given a notification if he or she is mind wandering for a longer period (e.g., longer than or equal to 30 seconds) by providing a feedback mechanism such as an auditory notification (e.g., “wake up”).

In some implementations, the techniques described herein can account for real-world environment 5 of the user 25 (e.g., visual qualities such as luminance, contrast, semantic context) in its evaluation of how much to modulate or adjust the presented content or feedback mechanisms to enhance the physiological response (e.g., pupillary response) of the user 25 to the visual characteristic 30 (e.g., feedback mechanism).

In some implementations, the physiological data (e.g., pupillary data 40) may vary in time and the techniques described herein may use the physiological data to detect a pattern. In some implementations, the pattern is a change in physiological data from one time to another time, and, in some other implementations, the pattern is series of changes in physiological data over a period of time. Based on detecting the pattern, the techniques described herein can identify a change in the stress level of the user (e.g., a high stress event) and can then provide a feedback mechanism (e.g., visual or auditory cue to focus on breathing) to the user 25 to return to an intended state (e.g., lower stress levels) during an experience. For example, a stress level of a user 25 may be identified by detecting a pattern in a user's gaze characteristic, heart rate, and/or PDA data, a visual or auditory cue associated with the experience may be adjusted (e.g., a feedback mechanism of a voice that states “focus on breathing” may further include a visual cue or a change in ambience of the scene), and the user's gaze characteristic, heart rate, and/or PDA data compared to the adjusted experience can be used to confirm the stress level of a user.

In some implementations, the techniques described herein can utilize a training or calibration sequence to adapt to the specific physiological characteristics of a particular user 25. In some implementations, the techniques present the user 25 with a training scenario in which the user 25 is instructed to interact with on-screen items (e.g., feedback objects). By providing the user 25 with a known intent or area of interest (e.g., via instructions), the techniques can record the user's physiological data (e.g., pupillary data 40) and identify a pattern associated with the user's physiological data. In some implementations, the techniques can change a visual characteristic 30 (e.g., a feedback mechanism) associated with content 20 in order to further adapt to the unique physiological characteristics of the user 25. For example, the techniques can direct a user to mentally select a button associated with an identified area in the center of the screen on the count of three and record the user's physiological data (e.g., pupillary data 40) to identify a pattern associated with the user's stress level. Moreover, the techniques can change or alter a visual characteristic associated with the feedback mechanism in order to identify a pattern associated with the user's physiological response to the altered visual characteristic. In some implementations, the pattern associated with the physiological response of the user 25 is stored in a user profile associated with the user and the user profile can be updated or recalibrated at any time in the future. For example, the user profile could automatically be modified over time during a user experience to provide a more personalized user experience (e.g., a personal educational experience for optimal learning experience while studying).

In some implementations, a machine learning model (e.g., a trained neural network) is applied to identify patterns in physiological data, including identification of physiological responses to presentation of content (e.g., content 20 of FIG. 1) during a particular experience (e.g., education, meditation, instructional, etc.). Moreover, the machine learning model may be used to match the patterns with learned patterns corresponding to indications of interest or intent of the user 25 to interact with the experience. In some implementations, the techniques described herein may learn patterns specific to the particular user 25. For example, the techniques may learn from determining that a peak pattern represents an indication of interest or intent of the user 25 in response to a particular visual characteristic 30 within the content and use this information to subsequently identify a similar peak pattern as another indication of interest or intent of the user 25. Such learning can take into account the user's relative interactions with multiple visual characteristics 30, in order to further adjust the visual characteristic 30 and enhance the user's physiological response to the experience and the presented content (e.g., focusing on particular areas of content versus other distracting areas).

In some implementations, the location and features of the head 27 of the user 25 (e.g., an edge of the eye, a nose or a nostril) are extracted by the device 10 and used in finding coarse location coordinates of the eyes 45 of the user 25, thus simplifying the determination of precise eye 45 features (e.g., position, gaze direction, etc.) and making the gaze characteristic(s) measurement more reliable and robust. Furthermore, the device 10 may readily combine the 3D location of parts of the head 27 with gaze angle information obtained via eye part image analysis in order to identify a given on-screen object at which the user 25 is looking at any given time. In some implementations, the use of 3D mapping in conjunction with gaze tracking allows the user 25 to move his or her head 27 and eyes 45 freely while reducing or eliminating the need to actively track the head 27 using sensors or emitters on the head 27.

By tracking the eyes 45, some implementations reduce the need to re-calibrate the user 25 after the user 25 moves his or her head 27. In some implementations, the device 10 uses depth information to track the pupil's 50 movement, thereby enabling a reliable present pupil diameter to be calculated based on a single calibration of user 25. Utilizing techniques such as pupil-center-corneal reflection (PCCR), pupil tracking, and pupil shape, the device 10 may calculate the pupil diameter, as well as a gaze angle of the eye 45 from a fixed point of the head 27, and use the location information of the head 27 in order to re-calculate the gaze angle and other gaze characteristic(s) measurements. In addition to reduced recalibrations, further benefits of tracking the head 27 may include reducing the number of light projecting sources and reducing the number of cameras used to track the eye 45.

In some implementations, the techniques described herein can identify a particular object within the content presented on the display 15 of the device 10 at a position in the direction of the user's gaze. Moreover, the techniques can change a state of the visual characteristic 30 associated with the particular object or the overall content experience responsively to a spoken verbal command received from the user 25 in combination with the identified stress level of the user 25. For example, a particular object within the content may be an icon associated with a software application, and the user 25 may gaze at the icon, say the word “select” to choose the application, and a highlighting effect may be applied to the icon. The techniques can then use further physiological data (e.g., pupillary data 40) in response to the visual characteristic 30 (e.g., a feedback mechanism) to further identify a stress level of the user 25 as a confirmation of the user's verbal command. In some implementations, the techniques can identify a given interactive item responsive to the direction of the user's gaze, and to manipulate the given interactive item responsively to physiological data (e.g., variability of the gaze characteristics). The techniques can then confirm the direction of the user's gaze based on further identifying stress levels of a user with physiological data in response to interactions with the experience (e.g., interacting within an intense video game). In some implementations, the techniques can remove an interactive item or object based on the identified interest or intent. In other implementations, the techniques can automatically capture images of the content at times when an interest or intent of the user 25 is determined.

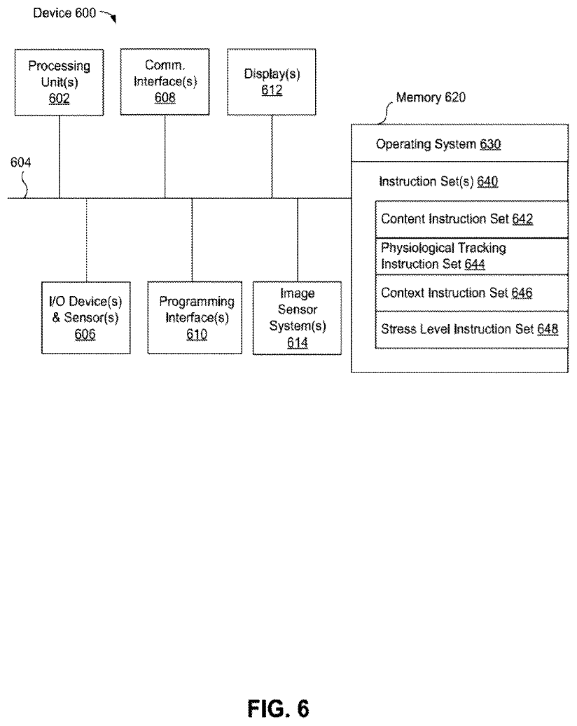

FIG. 6 is a block diagram of an example device 600. Device 600 illustrates an exemplary device configuration for device 10. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 10 includes one or more processing units 602 (e.g., microprocessors, ASICs, FPGAs, CPUs, CPUs, processing cores, and/or the like), one or more input/output (I/O) devices and sensors 606, one or more communication interfaces 608 (e.g., USB, FIREWIRE, THUNDERBOLT, IEEE 802.3x, IEEE 802.11x, IEEE 802.16x, GSM, CDMA, TDMA, GPS, IR, BLUETOOTH, ZIGBEE, SPI, I2C, and/or the like type interface), one or more programming (e.g., I/O) interfaces 610, one or more displays 612, one or more interior and/or exterior facing image sensor systems 614, a memory 620, and one or more communication buses 604 for interconnecting these and various other components.

In some implementations, the one or more communication buses 604 include circuitry that interconnects and controls communications between system components. In some implementations, the one or more I/O devices and sensors 606 include at least one of an inertial measurement unit (IMU), an accelerometer, a magnetometer, a gyroscope, a thermometer, one or more physiological sensors (e.g., blood pressure monitor, heart rate monitor, blood oxygen sensor, blood glucose sensor, etc.), one or more microphones, one or more speakers, a haptics engine, one or more depth sensors (e.g., a structured light, a time-of-flight, or the like), and/or the like.

In some implementations, the one or more displays 612 are configured to present a view of a physical environment or a graphical environment to the user. In some implementations, the one or more displays 612 correspond to holographic, digital light processing (DLP), liquid-crystal display (LCD), liquid-crystal on silicon (LCoS), organic light-emitting field-effect transitory (OLET), organic light-emitting diode (OLED), surface-conduction electron-emitter display (SED), field-emission display (FED), quantum-dot light-emitting diode (QD-LED), micro-electromechanical system (MEMS), and/or the like display types. In some implementations, the one or more displays 612 correspond to diffractive, reflective, polarized, holographic, etc. waveguide displays. In one example, the device 10 includes a single display. In another example, the device 10 includes a display for each eye of the user.

In some implementations, the one or more image sensor systems 614 are configured to obtain image data that corresponds to at least a portion of the physical environment 5. For example, the one or more image sensor systems 614 include one or more RGB cameras (e.g., with a complimentary metal-oxide-semiconductor (CMOS) image sensor or a charge-coupled device (CCD) image sensor), monochrome cameras, IR cameras, depth cameras, event-based cameras, and/or the like. In various implementations, the one or more image sensor systems 614 further include illumination sources that emit light, such as a flash. In various implementations, the one or more image sensor systems 614 further include an on-camera image signal processor (ISP) configured to execute a plurality of processing operations on the image data.

The memory 620 includes high-speed random-access memory, such as DRAM, SRAM, DDR RAM, or other random-access solid-state memory devices. In some implementations, the memory 620 includes non-volatile memory, such as one or more magnetic disk storage devices, optical disk storage devices, flash memory devices, or other non-volatile solid-state storage devices. The memory 620 optionally includes one or more storage devices remotely located from the one or more processing units 602. The memory 620 includes a non-transitory computer readable storage medium.

In some implementations, the memory 620 or the non-transitory computer readable storage medium of the memory 620 stores an optional operating system 630 and one or more instruction set(s) 640. The operating system 630 includes procedures for handling various basic system services and for performing hardware dependent tasks. In some implementations, the instruction set(s) 640 include executable software defined by binary information stored in the form of electrical charge. In some implementations, the instruction set(s) 640 are software that is executable by the one or more processing units 602 to carry out one or more of the techniques described herein.

The instruction set(s) 640 include a content instruction set 642, a physiological tracking instruction set 644, a context instruction set 646, and a stress level instruction set 648. The instruction set(s) 640 may be embodied a single software executable or multiple software executables.

In some implementations, the content instruction set 642 is executable by the processing unit(s) 602 to provide and/or track content for display on a device. The content instruction set 642 may be configured to monitor and track the content overtime (e.g., during an experience such as an education session) and/or to identify change events that occur within the content. In some implementations, the content instruction set 642 may be configured to inject change events into content (e.g., feedback mechanisms) using one or more of the techniques discussed herein or as otherwise may be appropriate. To these ends, in various implementations, the instruction includes instructions and/or logic therefor, and heuristics and metadata therefor.

In some implementations, the physiological tracking instruction set 644 is executable by the processing unit(s) 602 to track a user's physiological attributes (e.g., EEG amplitude/frequency, pupil modulation, eye gaze saccades, heart rate, EDA data, etc.) using one or more of the techniques discussed herein or as otherwise may be appropriate. To these ends, in various implementations, the instruction includes instructions and/or logic therefor, and heuristics and metadata therefor.

In some implementations, the context instruction set 646 is executable by the processing unit(s) 602 to determine a context of the experience and/or the environment (e.g., create a scene understanding to determine the objects or people in the content or in the environment, where the user is, what the user is watching, etc.) using one or more of the techniques discussed herein (e.g., object detection, facial recognition, etc.) or as otherwise may be appropriate. To these ends, in various implementations, the instruction includes instructions and/or logic therefor, and heuristics and metadata therefor.

In some implementations, the stress level instruction set 648 is executable by the processing unit(s) 602 to assess the stress level (e.g., high stress, low stress, etc.) of a user based on physiological data (e.g., eye gaze response) and context data of the content and/or environment using one or more of the techniques discussed herein or as otherwise may be appropriate. To these ends, in various implementations, the instruction includes instructions and/or logic therefor, and heuristics and metadata therefor.

Although the instruction set(s) 640 are shown as residing on a single device, it should be understood that in other implementations, any combination of the elements may be located in separate computing devices. Moreover, FIG. 6 is intended more as functional description of the various features which are present in a particular implementation as opposed to a structural schematic of the implementations described herein. As recognized by those of ordinary skill in the art, items shown separately could be combined and some items could be separated. The actual number of instructions sets and how features are allocated among them may vary from one implementation to another and may depend in part on the particular combination of hardware, software, and/or firmware chosen for a particular implementation.

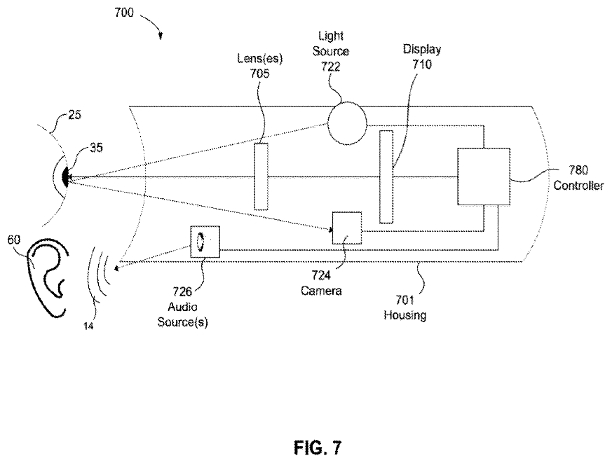

FIG. 7 illustrates a block diagram of an exemplary head-mounted device 700 in accordance with some implementations. The head-mounted device 700 includes a housing 701 (or enclosure) that houses various components of the head-mounted device 700. The housing 701 includes (or is coupled to) an eye pad (not shown) disposed at a proximal (to the user 25) end of the housing 701. In various implementations, the eye pad is a plastic or rubber piece that comfortably and snugly keeps the head-mounted device 700 in the proper position on the face of the user 25 (e.g., surrounding the eye of the user 25).

The housing 701 houses a display 710 that displays an image, emitting light towards or onto the eye of a user 25. In various implementations, the display 710 emits the light through an eyepiece having one or more lenses 705 that refracts the light emitted by the display 710, making the display appear to the user 25 to be at a virtual distance farther than the actual distance from the eye to the display 710. For the user 25 to be able to focus on the display 710, in various implementations, the virtual distance is at least greater than a minimum focal distance of the eye (e.g., 8 cm). Further, in order to provide a better user experience, in various implementations, the virtual distance is greater than 1 meter.

The housing 701 also houses a tracking system including one or more light sources 722, camera 724, and a controller 780. The one or more light sources 722 emit light onto the eye of the user 25 that reflects as a light pattern (e.g., a circle of glints) that can be detected by the camera 724. Based on the light pattern, the controller 780 can determine an eye tracking characteristic of the user 25. For example, the controller 780 can determine a gaze direction and/or a blinking state (eyes open or eyes closed) of the user 25. As another example, the controller 780 can determine a pupil center, a pupil size, or a point of regard. Thus, in various implementations, the light is emitted by the one or more light sources 722, reflects off the eye of the user 25, and is detected by the camera 724. In various implementations, the light from the eye of the user 25 is reflected off a hot mirror or passed through an eyepiece before reaching the camera 724.

The housing 701 also houses an audio system that includes one or more audio source(s) 726 that the controller 780 can utilize for providing audio to the user ears 60 via sound waves 14 per the techniques described herein. For example, audio source(s) 726 can provide sound for both background sound and the feedback mechanism that can be presented spatially in a 3D coordinate system. The audio source(s) 726 can include a speaker, a connection to an external speaker system such as headphones, or an external speaker connected via a wireless connection.

The display 710 emits light in a first wavelength range and the one or more light sources 722 emit light in a second wavelength range. Similarly, the camera 724 detects light in the second wavelength range. In various implementations, the first wavelength range is a visible wavelength range (e.g., a wavelength range within the visible spectrum of approximately 400-700 nm) and the second wavelength range is a near-infrared wavelength range (e.g., a wavelength range within the near-infrared spectrum of approximately 700-1400 nm).

In various implementations, eye tracking (or, in particular, a determined gaze direction) is used to enable user interaction (e.g., the user 25 selects an option on the display 710 by looking at it), provide foveated rendering (e.g., present a higher resolution in an area of the display 710 the user 25 is looking at and a lower resolution elsewhere on the display 710), or correct distortions (e.g., for images to be provided on the display 710).

In various implementations, the one or more light sources 722 emit light towards the eye of the user 25 which reflects in the form of a plurality of glints.

In various implementations, the camera 724 is a frame/shutter-based camera that, at a particular point in time or multiple points in time at a frame rate, generates an image of the eye of the user 25. Each image includes a matrix of pixel values corresponding to pixels of the image which correspond to locations of a matrix of light sensors of the camera. In implementations, each image is used to measure or track pupil dilation by measuring a change of the pixel intensities associated with one or both of a user's pupils.

In various implementations, the camera 724 is an event camera including a plurality of light sensors (e.g., a matrix of light sensors) at a plurality of respective locations that, in response to a particular light sensor detecting a change in intensity of light, generates an event message indicating a particular location of the particular light sensor.

It will be appreciated that the implementations described above are cited by way of example, and that the present invention is not limited to what has been particularly shown and described hereinabove. Rather, the scope includes both combinations and sub combinations of the various features described hereinabove, as well as variations and modifications thereof which would occur to persons skilled in the art upon reading the foregoing description and which are not disclosed in the prior art.

As described above, one aspect of the present technology is the gathering and use of physiological data to improve a user's experience of an electronic device with respect to interacting with electronic content. The present disclosure contemplates that in some instances, this gathered data may include personal information data that uniquely identifies a specific person or can be used to identify interests, traits, or tendencies of a specific person. Such personal information data can include physiological data, demographic data, location-based data, telephone numbers, email addresses, home addresses, device characteristics of personal devices, or any other personal information.

The present disclosure recognizes that the use of such personal information data, in the present technology, can be used to the benefit of users. For example, the personal information data can be used to improve interaction and control capabilities of an electronic device. Accordingly, use of such personal information data enables calculated control of the electronic device. Further, other uses for personal information data that benefit the user are also contemplated by the present disclosure.

The present disclosure further contemplates that the entities responsible for the collection, analysis, disclosure, transfer, storage, or other use of such personal information and/or physiological data will comply with well-established privacy policies and/or privacy practices. In particular, such entities should implement and consistently use privacy policies and practices that are generally recognized as meeting or exceeding industry or governmental requirements for maintaining personal information data private and secure. For example, personal information from users should be collected for legitimate and reasonable uses of the entity and not shared or sold outside of those legitimate uses. Further, such collection should occur only after receiving the informed consent of the users. Additionally, such entities would take any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices.

Despite the foregoing, the present disclosure also contemplates implementations in which users selectively block the use of, or access to, personal information data. That is, the present disclosure contemplates that hardware or software elements can be provided to prevent or block access to such personal information data. For example, in the case of user-tailored content delivery services, the present technology can be configured to allow users to select to “opt in” or “opt out” of participation in the collection of personal information data during registration for services. In another example, users can select not to provide personal information data for targeted content delivery services. In yet another example, users can select to not provide personal information, but permit the transfer of anonymous information for the purpose of improving the functioning of the device.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, content can be selected and delivered to users by inferring preferences or settings based on non-personal information data or a bare minimum amount of personal information, such as the content being requested by the device associated with a user, other non-personal information available to the content delivery services, or publicly available information.

In some embodiments, data is stored using a public/private key system that only allows the owner of the data to decrypt the stored data. In some other implementations, the data may be stored anonymously (e.g., without identifying and/or personal information about the user, such as a legal name, username, time and location data, or the like). In this way, other users, hackers, or third parties cannot determine the identity of the user associated with the stored data. In some implementations, a user may access his or her stored data from a user device that is different than the one used to upload the stored data. In these instances, the user may be required to provide login credentials to access their stored data.

Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter.

Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general purpose computing apparatus to a specialized computing apparatus implementing one or more implementations of the present subject matter. Any suitable programming, scripting, or other type of language or combinations of languages may be used to implement the teachings contained herein in software to be used in programming or configuring a computing device.

Implementations of the methods disclosed herein may be performed in the operation of such computing devices. The order of the blocks presented in the examples above can be varied for example, blocks can be re-ordered, combined, or broken into sub-blocks. Certain blocks or processes can be performed in parallel.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various objects, these objects should not be limited by these terms. These terms are only used to distinguish one object from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “or” as used herein refers to and encompasses any and all possible combinations of one or more of the associated listed items. It will be further understood that the terms “comprises” or “comprising,” when used in this specification, specify the presence of stated features, integers, steps, operations, objects, or components, but do not preclude the presence or addition of one or more other features, integers, steps, operations, objects, components, or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

The foregoing description and summary of the invention are to be understood as being in every respect illustrative and exemplary, but not restrictive, and the scope of the invention disclosed herein is not to be determined only from the detailed description of illustrative implementations but according to the full breadth permitted by patent laws. It is to be understood that the implementations shown and described herein are only illustrative of the principles of the present invention and that various modification may be implemented by those skilled in the art without departing from the scope and spirit of the invention.

1. A method of cognitive state assessment, the method comprising:
at a device comprising a processor:
obtaining physiological data associated with a user during an experience in an environment;
determining a context of the experience based on sensor data of the environment;
determining a stress level of the user during a portion of the experience based on the obtained physiological data and the context of the experience; and
providing a feedback mechanism based on the stress level.

2. The method of claim 1, wherein determining a stress level of the user during the portion of the experience further comprises determining a type of stress of the user based on the sensor data, and providing the feedback mechanism during the experience is further based on the type of stress.

3. The method of claim 1, further comprising:
providing a notification to the user based on the stress level; and
customizing content included in the experience based on the stress level of the user.

4. (canceled)

5. The method of claim 1, wherein the stress level is a first stress level, the method further comprising:
obtaining, using a sensor, first physiological data associated with a physiological response of the user to the feedback mechanism; and
determining a second stress level of the user based on the physiological response of the user to the feedback mechanism.

6. The method of claim 5, further comprising:
assessing the second stress level of the user based on the physiological response of the user to the feedback mechanism; and
determining whether the feedback mechanism reduced stress of the user by comparing the second stress level to the first stress level.

7. The method of claim 1, wherein determining the context of the experience includes:
generating a scene understanding of the environment based on the sensor data of the environment, the scene understanding comprising visual or auditory attributes of the environment; and
determining the context of the experience based on the scene understanding of the environment.

8. The method of claim 7, wherein the sensor data comprises image data, and generating the scene understanding is based at least on performing semantic segmentation of the image data and detecting one or more objects within the environment based on the semantic segmentation.

9. The method of claim 7, wherein determining the context of the experience comprises determining an activity of the user or a location of the user in a physical environment based on the scene understanding of the environment.

10. (canceled)

11. The method of claim 7, wherein determining the context of the experience comprises determining that an object is near the user in a physical environment based on the scene understanding.

12. The method of claim 1, wherein determining the context of the experience comprises identifying an attribute of the environment separate from content being presented to the user.

13. The method of claim 1, wherein determining the context of the experience comprises determining that a stimulus in the environment is associated with the stress level of the user, wherein the stimulus is separate from content being presented.

14. The method of claim 1, wherein the sensor data includes location data of the user, and determining the context of the experience includes determining a location of the user within the environment based on the location data.

15. The method of claim 1, wherein determining the context of the experience comprises determining an activity of the user based on a user's schedule.

16. The method of claim 1, wherein determining the context of the experience comprises determining that the user is consuming food.

17. The method of claim 15, wherein the stress level and the feedback mechanism are determined based on the user consuming food.

18. The method of claim 1, wherein the physiological data includes at least one of skin temperature, respiration, photoplethysmogram (PPG), electrodermal activity (EDA), eye gaze tracking, and pupillary movement that is associated with the user.

19. The method of claim 1, wherein the stress level is assessed using a statistical or machine learning-based classification technique.

20. The method of claim 1, further comprising: identifying the portion of the experience associated with stress level.

21-25. (canceled)

26. A device comprising:
a non-transitory computer-readable storage medium; and
one or more processors coupled to the non-transitory computer-readable storage medium, wherein the non-transitory computer-readable storage medium comprises program instructions that, when executed on the one or more processors, cause the one or more processors to perform operations comprising:
obtaining physiological data associated with a user during an experience in an environment; determining a context of the experience based on sensor data of the environment;
determining a stress level of the user during a portion of the experience based on the obtained physiological data and the context of the experience; and
providing a feedback mechanism based on the stress level.

27. (canceled)

28. A non-transitory computer-readable storage medium, storing program instructions executable on a device to perform operations comprising:
obtaining physiological data associated with a user during an experience in an environment; determining a context of the experience based on sensor data of the environment;
determining a stress level of the user during a portion of the experience based on the obtained physiological data and the context of the experience; and
providing a feedback mechanism based on the stress level.
